(19) United States
(12) Patent Application Publication
(10) Pub. No.: US 2004/0064011 A1
(43) Pub. Date: Apr. 1, 2004

(54) Seed for brachytherapy in different medical applications

(76) Inventors: Udo Grabowy, Emskirchen-Flamersheim (DE); Heinz Busch, Konigswinter-Thomasberg (DE)

Correspondence Address: MERCHANT & GOULD PC, P.O. BOX 2903, MINNEAPOLIS, MN, 55402-0903, US

(21) Appl. No.: 10/467,729

(22) PCT Filed: Feb. 14, 2002
(86) PCT No.: PCT/EP02/01543

(30) Foreign Application Priority Data
Feb. 14, 2001 (DE) 10107105.1

(51) Int. Cl. A61N 5/00

(52) U.S. Cl. 600/8

(57) ABSTRACT

The invention relates to an activatable seed consisting of at least one cylindrical body comprised of: a) a metal, for example metallic palladium, a palladium compound, a composite material made of a Pd compound and metal or mixtures thereof, optionally combined with b) a metallic material not containing Pd, wherein the palladium contained in a) contains Pd102.

The development of nuclear reactors and particle accelerators has allowed the production of approximately 2,500 isotopes. Approximately 300 thereof have a half-life of between 10 days and 100 years. Approximately 10 thereof are radioactive isotopes which are used in clinical brachytherapy. Numerous radionuclides are suitable for insertion or implantation in the body of the cancer patient.

The therapeutic benefit of radiation therapy reaches back 100 years. It was used at first for skin treatment. With the increasing availability of higher accelerating voltages, X-rays and electron rays were capable of reaching tumors located deeper in the body. In radiation therapy of tumor tissue (radiooncology), the cell-damaging effect is used for the purposeful destruction of tumor cells. In order to minimize radiation damage in healthy tissue during percutaneous radiation, the tumor is treated from different directions with a well-focused ray. Modern radiation systems are capable of processing a radiation profile which is precise within a few millimeters and is adjusted to the respective tumor.

Another way to protect the healthy tissue is used in brachytherapy (Greek brachy: close). A short-range, radiation emitter (range in the mm region) is introduced either directly into the tumor tissue interstitially or in very close vicinity (intracavitary) either permanently or for a specific period of time. One example is the treatment of the carcinoma of the prostate by the implantation of seeds. They contain a radionuclide with a typical half-life of a few weeks whose therapeutically effective radiation dose is limited to a few mm of the ambient tissue.

For a long time the naturally occurring radium isotope ²²⁶Ra was the only radionuclide that was available in sufficient quantity and purity for medical purposes. Due to the safety risk with a gaseous source it has been replaced in the meantime by other, currently available artificial radionuclides. The radiation sources which are mainly used nowadays are shown in Tab. 2.

TABLE 1
The most important radionuclides for implants; Ph means photons,
i.e. g- or X-radiation. ⁹⁰Sr and ¹⁸⁸W are the precursors
of ⁹⁰Y or ¹⁸⁸Re. ²²⁶Ra is stated for comparison reasons
with its two a-energies.

| | | | | | Ph energy | |
|---|---|---|---|---|---|---|
| | | Type of | β-energy | | (MeV) | |
| Radionuclide | Half-life | disintegration | mean | max. | mean | max. |
| ³²P | 14.3 days | β⁻ | 0.69 | 1.71 | | |
| ⁹⁰Sr/Y | 28.6 years | β⁻ | 0.17 | 0.55 | | |
| ⁹⁰Y | 64.1 hours | β⁻ | 0.92 | 2.27 | | |
| ¹⁰³Pd | 17.0 days | Ph | | | 0.020 | 0.023 |
| ¹²⁵I | 59.4 days | Ph | | | 0.032 | 0.035 |
| ¹⁸⁸W/Re | 69.4 days | β⁻, Ph_{—} | 0.16 | 0.35 | 0.21 | 0.29 |
| ¹⁸⁸Re | 16.9 hours | β⁻, Ph_{—} | 0.77 | 2.12 | 0.16 | 0.93 |
| ¹⁹²Ir | 73.8 days | β⁻, Ph_{—} | 0.17 | 0.67 | 0.37 | 1.06 |
| ²²⁶RA | 1,602 years | α, _Ph | 4.60⁹ | 4.78^{§} | 0.19 | 0.19 |

Most of the radionuclides mentioned above can be produced by means of neutron capture reaction in a reactor (reactor isotopes). Maximum or saturation activity is reached after approximately 5 half-lives in the neutron flux. One half-life is sufficient however for reaching half this activity. One example is ³²P which can be produced by the activation of stable ³¹P. Due to the relatively small capture cross section of 0.16 bam, an economic production is only viable in a high-flux reactor with a thermal neutron flux of over 10¹⁴ n/cm²s.

⁹⁰Sr on the other hand can be obtained as a fission product from spent reactor fuel elements. After approximately two weeks it stands with its similarly unstable decay product ⁹⁰Y in the so-called secular activity balance. ⁹⁰Y can also be produced directly by neutron capture from ⁸⁹Y, like ¹⁰³Pd from ¹⁰²Pd and ¹⁹²Ir from 191Ir. In the production of ¹²⁵I, ¹²⁴Xe is converted at first in the neutron flux into ¹²⁵Xe, which then decays further into ¹²⁵I with a half-life of 17.1 hours by electron capture.

Alternatively, radio nuclides can also be produced via a nuclear reaction, induced by high-energy particle bombardment. For this purpose a suitable stable isotope is bombarded with high-energy protons, deuterons or α-particles which are mostly accelerated with a cyclotron (cyclotron isotopes). ¹⁰³Pd can thus be produced via the reaction ¹⁰³Rh(p,n) ¹⁰³Pd. High-energy resonances in the effective cross section are exploited for this purpose.

With respect to their life, radionuclides differ especially in the type of radiation. There are pure β⁻ or _γ or X-ray emitters and mixed radiation emitters. Electrons have a considerably shorter range than _γ radiation of the same energy. This has far-reaching consequences both for radiation therapy as well as for dealing with such radiation emitters.

TABLE 2
Mean range of α⁻_{—} and _β radiation or half-thickness
for γ radiation in different energies in water and lead.

| | | | |
|---|---|---|---|
| | α, _mean | β, _mean | γ, _half-value |
| Energy | range [μm] | range [mm] | thickness [cm] |

| [MeV] | Water | Water | Lead | Water | Lead |
|---|---|---|---|---|---|
| 0.01 | 0.23 | 0.004 | | 1.33 | 0.005 |
| 0.1 | 1.39 | 0.14 | 0.01 | 4.06 | 0.11 |
| 0.2 | 2.10 | 0.43 | 0.03 | 5.06 | 0.59 |
| 0.5 | 3.60 | 1.71 | 0.12 | 7.14 | 0.68 |
| 1.0 | 5.88 | 4.32 | 0.31 | 9.90 | 0.84 |
| 5.0 | 36.88 | 22.5 | 1.45 | 22.8 | 1.36 |

As a result of the considerably steeper radial drop in dose, a ³²P seed requires less than 1/30 of the activity in comparison with a ¹²⁵I radiation emitter in order to deposit the same total dose in a distance of 2 mm.

The respective methods for producing radioactive implants use one of the described methods for producing the radionuclides. For producing seeds by using the ¹⁰³Pd radiation emitter, the radiation emitter is preferably produced with the help of the aforementioned nuclear reaction by proton bombardment. Subsequently, the radiating material is chemically processed and bound to a carrier substance.

In the production of the ¹⁰³Pd radiation emitter by neutron activation, a high share of radiating impurities is obtained by the silver isotopes ^{110m}Ag and ¹¹¹Ag in the case of natural isotope distribution of the used palladium, which impurities can merely be removed by chemical processing after the activation.

A radioopaque marker is embedded in the seed volume to ensure that the seeds can be recognized well in ultrasonic sound or X-ray light.

A number of methods for producing seeds have been described in the U.S. Pat. No. 4,994,013, U.S. Pat. No. 5,163,896, WO97/19706, EP-A-1,008995, WO86/04248.

The common aspect of all these methods is that relevant parts of the production sequence occur with open radioactivity or radioopaque markers, so-called hot processes. Only at the end of the production sequence will the radiation source be enclosed together with the radioopaque marker in a capsule which consists of titanium for example and has a typical wall thickness of 50 μm. Special safety precautions are necessary in practice in order to avoid direct contact with the radioactive material.

Depending on the design of the seed, the absorption is between 30 and 40% in the ¹⁰³Pd radiation emitter (≈22 keV). With respect to this absorption of the radiation it is necessary to dimension the quantity of the radiation material within the capsule in a respectively large way in order to achieve the desired emission. The capsule material will therefore be chosen under the aspect of minimizing the shielding.

It is the object of the present invention to avoid the disadvantages of the production methods previously employed by an alternative method. The seed is made completely from non-radioactive materials and activated only in a last work step. The activation occurs by neutron bombardment in a high-flux nuclear reactor. This means that materials used in the seed must be stable for use in such a reactor and should further respond in a neutral manner with the exception of the desired isotope. This method is known as a cold process. It is mandatory in this case to use especially pure materials, because even minute impurities can lead to undesirable radiation emitters. These conditions very strongly limit the choice of possible materials.

In a further embodiment, which could be referred to as a hot process, a radiation emitter can be built into the seed capsule during the production process. In contrast to the above method, merely the low absorption in comparison with conventional seeds is utilized in this application.

A radiation source (seed) is thus to be provided for radiation therapy, with a reduced quantity of radioactive material which similarly produces a sufficient therapeutic effect and is resistant to mechanical strain and bodily fluids. The surface of the seed should be provided with properties with favorable bodily compatibility after the decay of the radioactivity. The production process should be provided so as to be less complex. In particular, the safety requirements during the production process are to be minimized.

This object is achieved by the independent claims.

The invention is thus based on an activatable seed, comprising a self-supporting cylindrical body. The body can consist of a metal, e.g. metallic palladium, a palladium compound or a mixture of palladium or a palladium compound and metals or metal oxides. The body can be arranged as a composite material, with the outside capsule consisting of the aforementioned combination of materials, whereas the inner region consists of a material which does not contain palladium and merely assumes a supporting function. It is also possible to have an inhomogeneous alloy of the seed body with a possibly higher palladium concentration in the outer seed surface.

The said palladium is enriched with ¹⁰²Pd of the natural isotope distribution up to 100%. Any palladium with a content of ¹⁰²Pd can be used, especially palladium which is depleted with the isotopes ¹⁰⁸Pd, ¹¹⁰Pd, which lead to the undesirable radioactive isotopes ^{110m}Ag and ¹¹¹Ag.

The invention is thus based on the objective of integrating palladium not yet radioactive into a carrier matrix.

According to a first version, the solution can be as follows: ¹⁰²Pd is enriched up to 20 to 30% und disturbing isotopes are depleted accordingly. Pd is alloyed into a material which is not activated when subjected to a neutron flux. Before activation, the seed is covered with an also non-activatable layer which is biologically compatible. Amorphous carbon is a potential candidate.

The advantage is that the entire production occurs with non-radioactive material. The activation only occurs in a last step. Radiation protection measures only need to be taken at that point.

The invention therefore makes the production process substantially unproblematic. As a result, there is no time pressure any more concerning a rapid performance of the production process.

The materials employed must be relatively pure in order to avoid unintended radioactivity.

Since despite the enrichment of ¹⁰²Pd the isotopes ¹⁰⁸Pd and ¹¹⁰Pd are present, an undesirable activation will occur in any case. This can be reduced, however, through a respective configuration of the reaction procedure, e.g. by choosing the correct dwell time in the reactor. It is also expected that ¹⁰²Pd will be on the market at a cost-effective price, so that the economic viability of the process will be given.

According to a second version of the invention it is proceeded as follows: A basic body made of a biologically compatible metal such as palladium, titanium or any other material is produced. Then a small quantity of highly enriched palladium 102 is activated in the reactor so that it becomes Pd 103. Finally, the activated Pd 103 is brought by way of electrolysis onto the surface of the thus produced seed where it can deploy its radiation effect.

Highly enriched palladium 102 (over 95%) can be stored over a prolonged period such as several weeks in a high-flux reactor. The non-active seeds are then electro-plated with the highly enriched and now activated Pd.

The invention is now explained in closer detail by reference to the enclosed drawings, wherein:

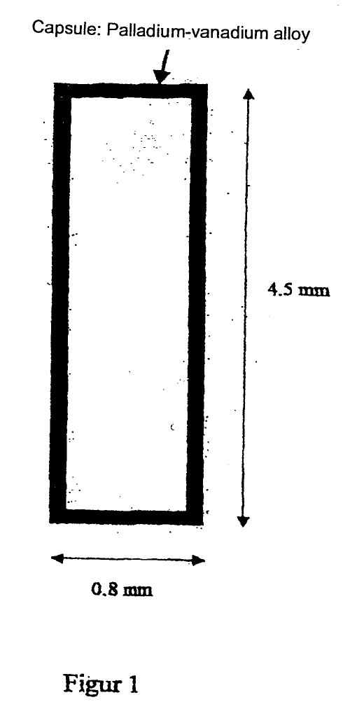

FIG. 1 shows a capsule;

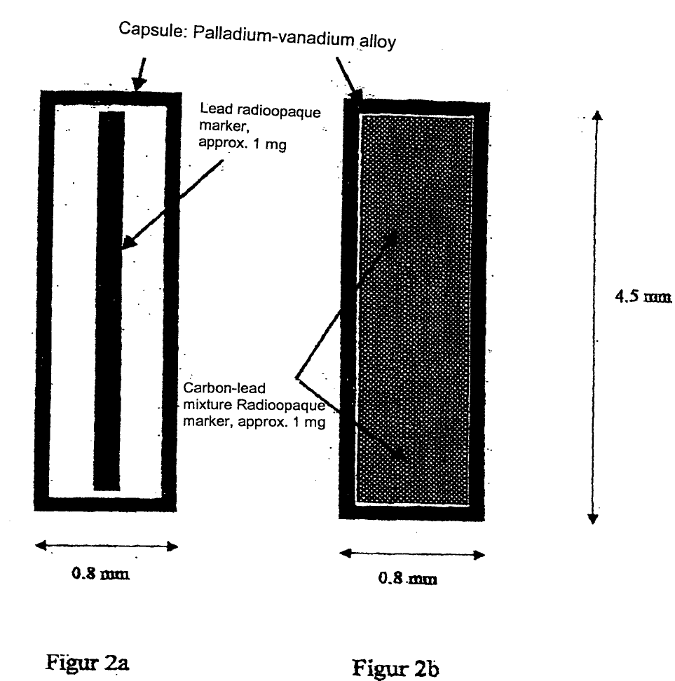

FIG. 2a shows a capsule with a rod-like radioopaque marker;

FIG. 2b shows a capsule with a filling material;

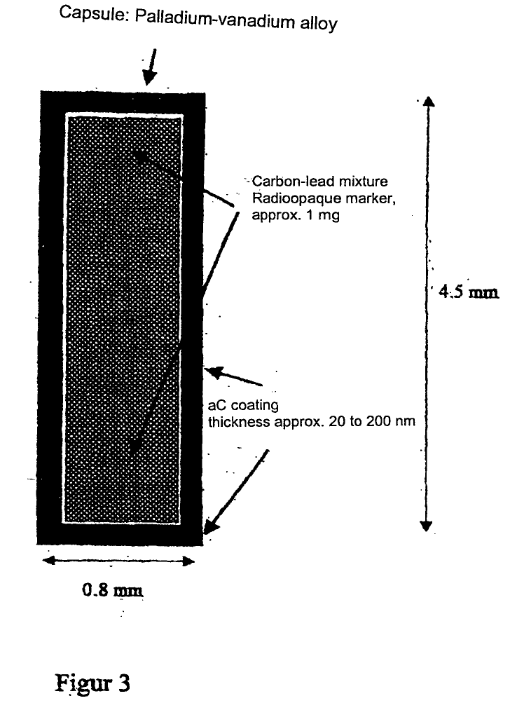

FIG. 3 shows a capsule with a material which contains a radioopaque marker.

The capsule described here is “self-supporting”, meaning that it does not require any special supporting or carrying construction.

Composite materials for producing such a seed body to which palladium is added are the metals Al, V and Ti, with vanadium being especially suitable.

In the neutron activation process the seed is subjected to a neutron flux in the nuclear reactor, with ¹⁰²Pd being converted into ¹⁰³Pd by capturing thermal neutrons. The amount of the conversion depends on the neutron flux and the dwell time in the nuclear reactor. In order to reach the saturation activity, the radiation period must exceed five half-times. This means more than 85 days for ¹⁰³Pd. After a time of three days, however, 11.5% of the possible activity has been reached. The situation is different in the case of the undesirable radio isotope ^{110m}Ag. This isotope is produced by neutron capture of ¹⁰⁸Pd to ¹⁰⁹Pd, subsequent β⁻ decay into ¹⁰⁹Ag, which on its parts is converted into ^{110m}Ag by neutron capture. As a result of this chain of events it takes approximately three days until a disturbing share of this undesirable activity is present. As a result of these conditions, a sufficiently high desired activity can be achieved within a period of approximately three days at a sufficiently high neutron flux (approx. 4*10¹⁴/cm*s) at low degrees of enrichment of ¹⁰²Pd, with the undesirable radiation being negligible.

The actual activity of the radioactive seed also depends on the share of the activatable precursor in the implant. The three parameters of quantity, neutron flux and radiation period can be varied independent from each other in order to set the actual activity which becomes therapeutically effective. The actual activity of the radioactive seed in accordance with the invention lies in the region of 0.1 μCi up to 300 mCi, better up to 50 mCi, and even more better up to 5 mCi.

The seed in accordance with the invention can comprise one or several coatings. The coatings can be applied by any kind of process, e.g. PVD, CVD, laser-induced CVD, plasma-activated CVD or thermal CVD, electrochemical coating, chemical coating such as precipitation, thermal spraying such as plasma spraying, depositing of metallic melts, dipping, immersing, plating, etc.

Any kind of coating material can be used. An intimate adherence to the capsule is desirable. Surface treatments can be considered for intensifying the adherence. The coating material should be corrosion-proof, resistant against radiation such as X-rays, neutrons, etc. during the activation and emission, und it should not be activated itself during the activation process. The coating should be shock-proof. Potential coating materials are amorphous carbon, plastic, glass, amorphous silicon, SiO₂, Al₂O₃, metals, metal alloys, nitrides, carbides, carbonitrides, as well as mixtures thereof.

The applied layer can have a thickness of 10 nm to 2 μm, preferably 20 to 100 nm, irrespective of the number of layers. The layer thickness can be used as a further parameter for this purpose to set the actual activity of the seed based on the X-radiation absorption of the coating material. Usually, the seed only comprises one single coating.

The coating or the first of several coatings can comprise an amorphous carbon with a thickness of 10 nm to 2 μm, preferably 20 to 100 nm. Such a coating adheres very well to the metallic capsule surface which consists of a metallic palladium and/or a compound thereof. The mechanical stability and the resistance against bodily fluids can be increased, especially in the case of long-term applications. The term “amorphous” means that deposited carbon does not have any regular crystalline structure.

According to a further embodiment, the marker is a central rod which is inserted in the inner space of the sleeve-like capsule. It can be fixed at its two ends by welding to the end caps. Tight clamping can also be considered.

The capsule best has such a shape that an even, homogeneous radiation field is established around the seed.

The capsule 1 shown in FIG. 1 comprises a sleeve-like part 2 as well as two semi-spherical caps 3.

The capsule according to FIG. 2 comprises a sleeve-like part 2 as well as flat, disk-like end caps 3 as well as a radioopaque marker 4.

The capsule 1 shown in FIG. 3 comprises a sleeve-like part 2 as well as end caps 3. The capsule 1 is filled with a filling material and a radioopaque marker in the form of a homogeneous particle mixture 4. The particle mixture preferably fills the entire interior space of the capsule.

The capsules according to the invention are preferably of an enclosed cylindrical shape. They have such a dimension that they can be transported with conventional apparatuses such as hypodermic syringes and needles into the body. The length is preferably 3 to 5 mm, preferably 4.5 mm. The outside diameter is 0.3 to 2.0 mm, preferably 0.8 mm. The wall thickness of the capsule is 10 to 250 μm, preferably 20 to 50 μm.

The seed can contain a radioopaque marker which is used for visualization under X-ray light. This marker can be held in the form of a thin core in the interior of the seed or can be filled as a mixture with a non-activatable material of low or high atomic number Z evenly in the interior of the seed. Lead or a lead compound is best used as a marker.

The method in accordance with the invention for producing an activatable seed comprises the following method steps:

a) A cylindrical body is produced. As already explained above, it consists of a metallic material such as metallic palladium or a palladium compound, or a composite of a palladium compound and a metal or of mixtures thereof, optionally in combination with a metallic material which is not palladium. The said palladium comprises Pd 102.

b) A radioopaque marker and/or a filling material is optionally filled into the body.

c) The body is optionally sealed off.

d) One or several coatings are optionally applied to the body.

In step (a) the capsule is produced completely, with the exception of the sealing of the capsule.

The activation can be performed in the presence of any neutron source which produces neutron rays in sufficient intensity. The duration of the activation process depends on the desired actual activity of the seed (cf. in this respect WO 86/04248). For the purpose of the invention, neutron fluxes of 1×10¹³ to 3×10¹⁵ (cm²s)⁻¹, preferably 1 to 20×10¹⁴ (cm²s)⁻¹, are preferred with durations of 1 to 10 days.

Usually, only a fraction of the Pd 102 present is converted. The finally obtained seed can contain both Pd 102 as well as Pd 103.

This offers the possibility of reactivating unused seeds after a decay period.

According to the invention, the activation can be performed after different steps of the production process. In a preferred embodiment, the activation is performed after the complete cold production of the seed according to step (d). This means that subsequently to the activation no further production step needs to be performed on the seed. The risk of unnecessary irradiation of the environment is thus minimized. This also offers the possibility of producing radioactive seeds on order by activating prefabricated seeds. A further advantage is that any undesirable degradation of Pd 103 prior to the therapeutic application is prevented. This is important with respect to the low half-life of Pd 103.

According to a further embodiment, the activation is performed after the complete production of the seed according to step (c) prior to any coating. Polymer coating materials can thus be used which otherwise do not withstand the activation conditions.

A number of examples are provided below:

### EXAMPLE I

This example relates to an activation process by using Pd with an enrichment of 90% with respect to Pd 102. Table 1 shows the required quantity of Pd and the respective percent by volume of Pd in the capsule material in order to produce an activity of 5 mCi at the start of the activation at a thermal flux of 4*10\hat{ }14 neutrons/sec*cm².

Seeds with a capsule of cylindrical shape were produced from an alloy of Pd and V, with a length of 4.5 mm, with an outside diameter of 0.8 mm and a wall thickness having the following values: i) 50 μm, ii) 40 μm, iii) 30 μm.

Table I: Degree of enrichment 90%

| Activation period | | 1 day | 3 days | 10 days |
|---|---|---|---|---|
| Total quantity of | | 0.67 mg | 0.23 mg | 0.08 mg |
| Pd in capsule | | | | |
| Percent by | i) wall thickness 50 μm | 9.6% | 3.2% | 1.1% |
| volume of Pd | ii) wall thickness 40 μm | 12% | 4.0% | 1.4% |
| with respect to | iii) wall thickness 30 μm | 16% | 5.3% | 1.9% |
| total volume of | | | | |
| the capsule | | | | |
| material | | | | |

According to Table I it is only necessary to produce a small quantity of the capsule material of Pd in order to produce an activity of 5 mCi, which is subject to the condition however that the amount of enrichment with respect to Pd 102 is 90%. The quantity of Pd can be reduced the further that the activation time is increased. In the case of a wall thickness of 30 μm and an activation period of 10 days, the share of material (volume) of vanadium is 98.1%, at an activation period of three days it is 94.7%, and 84% for one day.

### EXAMPLE II

Example I was repeated in example II, with the exception of the fact that the degree of enrichment with respect to Pd 102 was 30%. The desired and measured activity was 5 mCi. The neutron flux was 4×10¹⁴ (cm²s)⁻¹. The capsule was of a sealed cylindrical shape with a length of 4.5 mm, an outside diameter of 0.8 mm and a wall thickness of i) 50 μm, ii) 40 μm, iii) 30 μm. V was used as the alloy element.

TABLE II
Degree of enrichment = 30%

| Activation period | | 1 day | 3 days | 10 days |
|---|---|---|---|---|
| Total quantity | | 2.0 mg | 0.67 mg | 0.24 mg |
| of Pd in capsule | | | | |
| Percent by | i) wall thickness 50 μm | 28.7% | 9.6% | 3.4% |
| volume of Pd | ii) wall thickness 40 μm | 35.9% | 12% | 4.2% |
| with respect to | iii) wall thickness 30 μm | 47.9% | 16% | 5.6% |
| total volume of | | | | |
| the capsule | | | | |
| material | | | | |

As can be seen from this example, the proportion of vanadium in the alloy is reduced due to the low Pd 102 enrichment.

### EXAMPLE III

Example III is again identical to example I, with the exception that the degree of enrichment with respect to Pd 102 was 10%.

TABLE III
Degree of enrichment = 10%

| Activation period | | 1 day | 3 days | 10 days |
|---|---|---|---|---|
| Total quantity | | 6.0 mg | 2.0 mg | 0.7 mg |
| of Pd in capsule | | | | |
| Percent by | i) wall thickness 50 μm | 86.2% | 28.7% | 10.1% |
| volume of Pd | ii) wall thickness 40 μm | — | 35.9% | 12.7% |
| with respect to | iii) wall thickness 30 μm | — | 47.9% | 16.9% |
| total volume of | | | | |
| the capsule | | | | |
| material | | | | |

According to table III the time period of activation of one single day is not sufficient for 40 μm and 30 μm capsules in order to produce an activity of 5 mCi, even when the entire capsule material is Pd. The activation period or the wall thickness of the housing must therefore be changed.

1. An activatable seed, consisting of a cylindrical body, comprising:
a) a metal such as metallic palladium, a palladium compound, a composite of a palladium compound and a metal or of mixtures thereof, optionally in combination with
b) a metallic material which does not contain any Pd, with the palladium contained in a) containing Pd 102.

2. A seed as claimed in claim 1, wherein the body consists of a palladium alloy with V, Ti, Al or mixtures thereof.

3. A seed as claimed in claim 1 or 2, characterized in that the capsule contains up to 100% palladium.

4. A seed as claimed in one of the preceding claims, characterized in that the body comprises one or several coatings.

5. A seed as claimed in claim 4, characterized in that a first coating comprises amorphous carbon and has a thickness of 10 nm to 2 μm, preferably 20 to 100 nm.

6. A seed as claimed in one of the claims 1 to 5, characterized in that a radioopaque marker is provided, comprising a metal of a high atomic number (Z), preferably Pb or Rh or compounds or alloys or mixtures thereof.

7. A seed as claimed in one of the claims 1 to 6, characterized in that the body has the following properties:
7.1 a length of 2.0 to 8.0 mm, preferably 4.5 mm;
7.2 an outside diameter of 0.1 to. 2.0 mm, preferably 0.8 mm;
7.3 a wall thickness of 10 to 250 μm, preferably 20 to 50 μm.

8. A seed as claimed in claim 7, comprising-the following features:
8.1 a radioopaque marker with a thickness of 0.1 to 0.8 mm, preferably 0.1 to 0.3 mm, and a length corresponding to the length of the seed;
8.2 one or several coatings, each of which has a thickness of 10 nm to 2 μm, preferably 20 to 100 nm.

9. A method for producing a seed in the following method steps:
9.1 palladium is incorporated into a carrier matrix which is not activated during the action of a neutron flux;
9.2 this seed is then also coated with a non-activatable layer;
9.3 the activation occurs in the neutron flux of a reactor after the coating with the non-activatable layer.

10. A method for producing a seed;
10.1 with a biologically compatible body being produced;
10.2 with highly enriched palladium 102 being activated in the reactor, so that palladium 103 is produced;
10.3 with the activated palladium 103 being brought to the surface of the seed by electrolytic or electroplating treatment.
